(19) United States
(12) Patent Application Publication
(10) Pub. No.: US 2024/0238999 A1
(43) Pub. Date: Jul. 18, 2024

(54) SOLID SHAVING AID COMPOSITION

(71) Applicant: BIC Violex Single Member S.A., Anoixi (GR)

(72) Inventors: Maria-Thiresia ZANIDAKI, Anoixi (GR); Georgios VLACHOS, Anoixi (GR)

(73) Assignee: BIC Violex Single Member S.A., Anoixi (GR)

(21) Appl. No.: 18/563,178

(22) PCT Filed: May 30, 2022
(86) PCT No.: PCT/EP2022/064520

§ 371 Date: Nov. 21, 2023

(30) Foreign Application Priority Data
May 31, 2021 (EP) 21176933.6

(51) Int. Cl. B26B 21/44; A61K 8/02; A61K 8/41; A61Q 9/02

(52) U.S. Cl. CPC B26B 21/44; A61K 8/0216; A61K 8/416; A61Q 9/02; A61K 2800/596; A61K 2800/87

(57) ABSTRACT

The present disclosure relates to a razor cartridge comprising a housing having a front edge and a rear edge; one or more shaving blades disposed between the front edge and the rear edge; and at least one shaving bar disposed in front of the blade(s) and/or rear of the blade(s) which comprises a solid shaving aid composition; wherein the solid shaving aid composition comprises, relative to the total weight of the solid shaving aid composition: at least about 40 wt.-%, of a wax having a melting point of at least about 40° C. and a solubility in water at about 25° C. of less than about 1000 mg/L; and at least about 8 wt.-% of a cationic surfactant.

### CROSS-REFERENCE TO RELATED APPLICATIONS

This is a National Phase application under 35 U.S.C. § 371 of International Application No. PCT/FR2022/064520, filed May 30, 2022, now published as WO 2022/253750 A1, which claims benefit to the European Patent Application No. 21176933.6, filed May 31, 2021, its content being incorporated herein by reference.

### TECHNICAL FIELD

The present disclosure relates to the field of razor cartridges. More specifically, the present disclosure relates to razor cartridges comprising solid shaving aid compositions.

### BACKGROUND

Razors generally include a handle and a razor cartridge fixedly or releasably attached to the handle. The razor cartridge includes at least one blade for shaving hair. The razor cartridge may also include a solid shaving aid composition, often in the shape of a shaving aid bar extending parallel to at least one blade. The user holds the handle and repeatedly moves the razor across an area of the body to be shaved, e.g., the face, until hair is removed from the surface of the body. The shaving aid bar may function to aid in shaving and to decrease or prevent skin irritation due to the repeated exposure of the skin to sharp razor blades.

Razor cartridges comprising such solid shaving aid compositions are known in the art, for instance from WO 2016/162080 A1. However, while successful in aiding with comfortable and simplified shaving, there is still room for improvement. For instance, current products still have limited durability, i.e. longer acceptance by the user. In particular, especially after multiple uses, razor cartridges comprising a solid shaving aid composition may exhibit reduced skin hydration, maneuverability and glide, leaving the user dissatisfied after a relatively low number of shaves.

### SUMMARY

Following diligent research, it was surprisingly found that a new formulation based on cationic surfactants is generally advantageous in providing razor cartridges having improved durability when combined with a wax.

According to a first aspect, the present disclosure relates to a razor cartridge comprising a housing having a front edge and a rear edge. The housing comprises one or more shaving blades disposed between the front edge and the rear edge. At least one shaving bar is disposed in front of the blade(s) and/or rear of the blade(s). The shaving bar comprises a solid shaving aid composition. The solid shaving aid composition may comprise, relative to the total weight of the solid shaving aid composition: at least about 8 wt.-% of a cationic surfactant and at least about 40 wt.-% of a wax having a melting point of at least about 40° C. and a solubility in water at about 25° C. of less than about 1000 mg/L.

In some embodiments the wax may comprise a co-surfactant, in particular a hydroxylated hydrocarbon-based wax.

In some embodiments the wax may comprise an ester wax, in particular a hydrocarbon-based ester wax.

In some embodiments the cationic surfactant may comprise a quaternary ammonium salt of general formula (I),

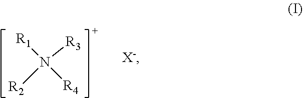

wherein:
X⁻ is an anion selected from halides, such as chloride, bromide and iodide; (C₁-C₆)alkyl sulfates, such as methyl sulfate; phosphates; alkyl, aryl, and alkylaryl sulfonates such as mesylate; and anions derived from carboxylic acids, such as acetate and lactate;
R₁ and R₂ are each independently from each other selected from optionally substituted linear or branched aliphatic residues comprising between 1 to about 4 carbon atoms and optionally substituted (hetero)aromatic residues such as (hetero)aryl comprising between about 5 to about 12 carbon atoms and alkyl(hetero)aryl residues comprising from about 6 to about 12 carbon atoms; and
R₃ is selected from optionally substituted linear or branched saturated or unsaturated alkyl residues comprising between about 8 and about 32 carbon atoms, specifically between about 10 and about 28 carbon atoms, and in particular between about 12 about 26 carbon atoms; and
R₄ is either defined as R₁ and R₂ or defined as R₃;
or wherein:
X⁻ is an anion selected from halides, such as chloride, bromide and iodide; (C₁-C₆)alkyl sulfates, such as methyl sulfate; phosphates; alkyl, aryl, and alkylaryl sulfonates such as mesylate; and anions derived from carboxylic acids, such as acetate and lactate;
R₁ and R₂ form an optionally substituted saturated, unsaturated, aromatic or heteroaromatic cyclic residue comprising between about 4 to about 24 carbon atoms, more specifically between about 4 and about 10 carbon atoms, and in particular between about 6 to about 16 carbon atoms; and
R₃ is absent and the bond to either R₁ and R₂ is a double bond, or is defined as R₁ and R₂, or represents an optionally substituted linear or branched alkyl residue comprising between 1 and about 6 carbon atoms, in particular between 1 and about 4 carbon atoms; and
R₄ is selected from optionally substituted linear or branched saturated or unsaturated alkyl residues comprising between about 8 and about 32 carbon atoms, specifically between about 10 and about 28 carbon atoms, and in particular between about 12 and about 26 carbon atoms.

In some embodiments, the cationic surfactant may comprise behentrimonium methosulfate.

In some embodiments, the solid shaving aid composition may further comprise a zwitterionic surfactant.

In some embodiments, the zwitterionic surfactant may comprise a betaine.

In some embodiments the cationic or zwitterionic surfactant may have a solubility in water of less than about 1000 mg/L, in particular less than about 100 mg/L and in particular less than about 1 mg/L, at about 25° C.

In some embodiments the solid shaving aid composition may comprise, relative to the total weight of the solid shaving aid composition: less than about 10 wt.-%, more specifically less than about 5 wt.-%, and in particular less than about 3 wt.-%, of monovalent linear or branched saturated or unsaturated carboxylic acid salts having between about 8 and about 24 carbon atoms, in particular between about 10 and about 20 carbon atoms; and/or less than about 10 wt.-%, more specifically less than about 5 wt.-%, and in particular less than about 3 wt.-%, of monovalent linear or branched saturated or unsaturated carboxylic acids having between about 8 and about 24 carbon atoms, in particular between about 10 and about 20 carbon atoms; and/or
less than about 10 wt.-%, more specifically less than about 5 wt.-%, and in particular less than about 3 wt.-%, of a combination of monovalent linear or branched saturated or unsaturated carboxylic acid salts having between about 8 and about 24 carbon atoms, in particular between about 10 and about 20 carbon atoms, and monovalent linear or branched saturated or unsaturated carboxylic acids having between about 8 and about 24 carbon atoms, in particular between about 10 and about 20 carbon atoms, less than about 10 wt.-%, more specifically less than about 5 wt.-%, and in particular less than about 3 wt.-%, of a fatty acid salt comprising a monovalent linear or branched saturated or unsaturated carboxylic acid salt having between about 8 and about 24 carbon atoms. In some embodiments, the solid shaving aid composition may be substantially free or free of such carboxylic acid salts and carboxylic acids.

In some embodiments the solid shaving aid composition may comprise, relative to the total weight of the solid shaving aid composition, less than about 4 wt.-%, more specifically less than about 2 wt.-%, and in particular less than about 0.5 wt.-%, of a silicone compound. In some embodiments, the solid shaving aid composition may be substantially free or free of silicone compounds.

In some embodiments the solid shaving aid composition may comprise a non-ionic surfactant.

In some embodiments the solid shaving aid composition may comprise one or more oils.

In some embodiments the solid shaving aid composition may further comprise one or more humectant oils having a molecular weight of greater than about 500 g/mol, more specifically a castor oil derivative, and in particular castoryl maleate.

In some embodiments the solid shaving aid composition may comprise, relative to the total weight of the solid shaving aid composition, between about 0.1 wt.-% and about 5 wt.-%, in particular between about 0.5 wt.-% and about 4.5 wt.-%, of a butter or a mixture of butters.

In another aspect, the present disclosure relates to a solid shaving aid composition having a composition as defined in the first aspect. In some embodiments, the solid shaving composition may be shaped into the form of a bar, more specifically a bar configured to be placed onto a razor cartridge, and in particular a bar having a length of between about 30 mm to about 80 mm, a width between about 2 mm and about 10 mm, and a depth of about 0.2 mm to about 4 mm.

In still another aspect, the present disclosure relates to the use of a solid shaving aid composition having a composition defined above in a multiplicity of facial or leg shaving operations, wherein the facial or leg shaving operations is repeated for at least about 8 times, specifically about 9 times, and in particular at least about 10 times.

### BRIEF DESCRIPTION OF THE DRAWINGS

FIG. 1 shows a razor cartridge including a housing including at least one shaving bar including a solid shaving aid composition, according to aspects of the disclosure.

### DETAILED DESCRIPTION

Hereinafter, a detailed description will be given of the present disclosure. The terms or words used in the description and the claims of the present disclosure are not to be construed limitedly as only having common-language or dictionary meanings and should, unless specifically defined otherwise in the following description, be interpreted as having their ordinary technical meaning as established in the relevant technical field. The detailed description will refer to specific embodiments to better illustrate the present disclosure, however, it should be understood that the presented disclosure is not limited to these specific embodiments.

As indicated above, it was found that a new formulation based on cationic or zwitterionic surfactants, and in particular based on cationic surfactants, is generally advantageous in providing razor cartridges having improved durability when combined with a wax. Panel tests of exemplary compositions according to the present disclosure showed that a razor cartridge comprising the shaving aid composition according to the present disclosure is endowed with increased durability and skin hydration, maneuverability and/or glide. These results will be further elaborated in the experimental section.

Without wishing to be bound by theory, it is believed that the combination of a relatively high amount of cationic surfactant with a waxy base component is providing the aforementioned benefit: The wax is present in relatively high amounts and has a melting point above skin temperature and a relatively low solubility in water. Thus, it contributes to the formation of a relatively water-insoluble solid base matrix for the dispersion of the cationic or zwitterionic surfactant and is believed—again without wishing to be bound by theory—to provide the basis of providing a slow release of surfactant actives during the shaving action. The solid shaving composition thereby facilitates providing a long lasting benefit to the user.

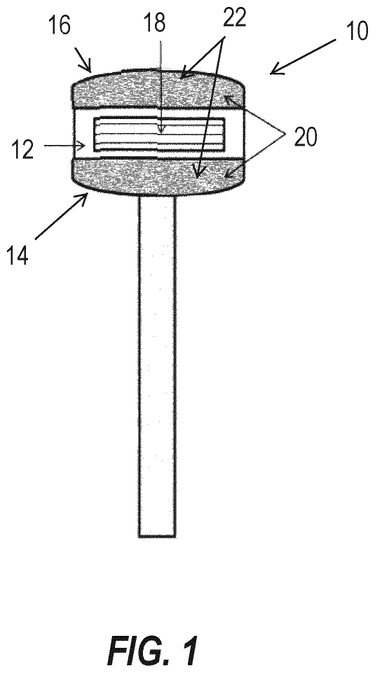

Accordingly, in a first aspect, and in particular with respect to FIG. 1, the present disclosure relates to a razor cartridge comprising a housing 12 having a front edge 14 and a rear edge 16. The housing 12 comprises one or more shaving blades 18 disposed between the front edge 14 and the rear edge 16. At least one shaving bar 20 is disposed in front of the blade(s) 18 and/or rear of the blade(s). The shaving bar 20 comprises a solid shaving aid composition 22. The solid shaving aid composition may comprise, relative to the total weight of the solid shaving aid composition: at least about 8 wt.-% of a cationic surfactant and at least about 40 wt.-%, of a wax having a melting point of at least about 40° C. and a solubility in water at about 25° C. of less than about 1000 mg/L.

The shaving bar 20 is not particularly limited and can have any suitable shape. Typically, it is in the form of at least one strip or another elongated body which is arranged substantially parallel to the shaving blades. The shaving bar 20 comprises the shaving aid composition 22. The shaving aid composition 22 is solid for the present purposes, i.e. solid in the sense that the shaving aid composition retains its shape once applied to the cartridge and does not migrate or “run off” under normal storage conditions (e.g. temperature of up to about 25° C. and a relative humidity of up to about 60%) and/or intended use conditions. However, it should be understood that the solid state of the shaving aid composition is not particularly limited and also includes wax-like characteristics. The shaving aid composition may be solid at room temperature, e.g. at about 25° C.

The solid shaving aid composition 22 may comprise at least about 40 wt.-% of a wax. The term “wax,” in the present disclosure is intended to be used as is well-established in the field of cosmetics and is, in particular, meant to refer to a lipophilic (fatty) compound that is solid at e.g. room temperature (e.g. about 25° C.) with a reversible solid/liquid change of state. The method of measuring the melting point of the wax is not particularly limited and may be measured using a differential scanning calorimeter (DSC), for example the calorimeter sold under the name DSC 3 by the company Mettler Toledo.

Amongst the aforementioned waxes, the present disclosure relates to waxes having a melting point of at least about 40° C. and a solubility water at about 25° C. of less than about 1000 mg/L. As stated above, these properties may help in providing a relatively water-insoluble solid base matrix for the dispersion of the cationic surfactant. In some embodiments, the wax may have a melting point of at least about 40° C., more specifically at least about 42° C. and in particular at least about 45° C. In some embodiments, the wax may have a melting point range of between about 40° C. and about 120° C., more specifically between about 42° C. and about 100° C., and in particular between about 45° C. and about 80° C.

In some embodiments, the wax may have a solubility in water at about 25° C. of less than about 100 mg/L, more specifically a solubility of less than about 10 mg/L, and in particular a solubility of less than about 1 mg/L. In some embodiments, the wax may be substantially insoluble or insoluble in water at about 25° C. The method of determining the solubility of the wax is not particularly limited and may be performed by any suitable means, for instance using a USP Dissolution Apparatus 2 (paddle type).

Examples of waxes include the following:

### a) Hydrocarbon-Based Wax

In some embodiments, the wax may be a hydrocarbon-based wax. The term “hydrocarbon-based wax” is intended to refer to a wax formed essentially from, or even constituted by, carbon and hydrogen atoms, and possibly oxygen and nitrogen atoms, and not containing any silicon or fluorine atoms. It may contain functional groups such as alcohol, ether, amine and/or amide groups.

In some embodiments, it may be advantageous that the hydrocarbon-based wax is a co-surfactant. The term “co-surfactant” in the present disclosure is intended to be used as is well-established in the field of cosmetics and is, in particular, meant to refer to a second (weaker) surfactant that is used in improve or contribute the performance of a primary surfactant (in this case: the cationic surfactant). A weaker co-surfactant has a lower surfactant effectiveness relative to the cationic surfactant when tested under otherwise identical conditions. Suitable testing conditions are well-known in the art. An exemplary and convenient measure of surfactant efficiency includes the amount of surfactant needed to reduce the surface tension by 20 mN/m at a surface age of 0.1 s, as disclosed in Miller, Tenside Surfactants Detergents 42(4):204-209; 2005, the disclosure of which is incorporated herein by reference. However, other tests may be used as well. One way in which a co-surfactant may improve or contribute the performance of a primary surfactant is that it facilitates the emulsification provided by the cationic surfactant, i.e. it acts as an emulsion stabilizer.

In context of the aforementioned it should be understood that the present disclosure does not intend to distinguish between tensides, surfactants, emulsifiers and emollients. All these compounds are endowed with a certain degree of surface-active activity and are used interchangeably unless expressly stated otherwise.

In some embodiments, in particular in cases where the hydrocarbon-based wax is a co-surfactant, it may be particularly advantageous that the hydrocarbon-based wax is a hydroxylated hydrocarbon-based wax.

In some embodiments, the hydrocarbon-based wax comprises a linear or branched, saturated or unsaturated, monovalent or multivalent hydrocarbyl alcohol having between about 8 and about 32 carbon atoms, specifically between about 10 and about 28 carbon atoms, and in particular between about 12 about 24 carbon atoms. More specifically, the wax may comprise a (in particular a linear) mono- or divalent alkyl alcohol having between about 8 and about 32 carbon atoms, specifically between about 10 and about 28 carbon atoms, and in particular between about 12 about 24 carbon atoms. In some embodiments, the wax may comprise a fatty alcohol, in particular cetyl alcohol, stearyl alcohol, behenyl alcohol, or mixtures thereof such as cetearyl alcohol. A commercial example includes Lanette O, sold by BASF SE, Germany. A fatty alcohol is the aforementioned sense may in particular be a monovalent linear alkyl alcohol having between about 8 and about 24 carbon atoms.

### b) Ester Wax

In some embodiments the wax may comprise an ester wax, in particular a hydrocarbon-based ester wax. The term “ester wax” in the present disclosure is intended to be used as is well-established in the field of cosmetics and is, in particular, meant to refer to a wax comprising at least one ester group.

In some embodiments, the ester wax is an ester of a C₈-₄₀-hydrocarbyl carboxylic acid, which may optionally be substituted with one or more heteroatoms such as O, N, S or P, with a C₂-C₈ polyol having between 2 and 8 hydroxyl groups. In some embodiments, the C₈-C₄₀-hydrocarbyl carboxylic acid may comprise at least one saturated or unsaturated C₁₀-C₃₂-moiety, more specifically at least one saturated or unsaturated C₁₂-C₂₆-moiety, and in particular at least one saturated or unsaturated C₁₄-C₂₄-moiety. In some embodiments, the C₂-C₈ polyol has between 2 and 6, between 2 and 4, between 3 and 6, or between 3 and 5 hydroxyl groups. In some embodiments, the ester wax is a monoester, a diester, a triester or a tetraester, and in particular a monoester or diester.

In some embodiments, it may be advantageous that the ester wax still comprises hydroxyl groups, i.e. that the ester is (formally) formed by reaction of less C₈-C₄₀-hydrocarbyl carboxylic acids than the number of hydroxyl groups in the C₂-C₈ polyol and/or that the C₈-C₄₀-hydrocarbyl carboxylic acids are substituted with hydroxyl groups. In some embodiments, it may thus be advantageous that the ester wax comprises one, two, three or four (free) hydroxyl groups. The remaining hydroxyl groups may have the advantage of providing co-surfactant performance to the ester wax.

In some embodiments, the ester wax is an ester of a C₈-C₄₀-hydrocarbyl alcohol, which may optionally be substituted with one or more heteroatoms such as O, N, S or P, with a C₂-C₈ carboxylic acid having between 2 and 6 carboxylic acid groups. In some embodiments, the C₈-C₄₀-hydrocarbyl alcohol may comprise at least one saturated or unsaturated C₁₀-C₃₂-moiety, more specifically at least one saturated or unsaturated C₁₂-C₂₆-moiety, and in particular at least one saturated or unsaturated C₁₄-C₂₄-moiety. In some embodiments, the C₂-C₈ carboxylic acid has between 2 and 6, between 2 and 4, between 3 and 6, or between 3 and 5 carboxylic acid groups. In some embodiments, the ester wax is a mono ester, a diester, a triester or a tetraester.

In some embodiments, it may be advantageous that the ester wax still comprises hydroxyl groups, i.e. that the ester is (formally) formed by reaction of more C₈-C₄₀-hydrocarbyl alcohols than the number of carboxylic acid groups in the C₂-C₈ carboxylic acid and/or that the C₈-C₄₀-hydrocarbyl alcohols are substituted with hydroxyl groups. In some embodiments, it may thus be advantageous that the ester wax comprises one, two, three or four (free) hydroxyl groups. The remaining hydroxyl groups may have the advantage of providing co-surfactant performance to the ester wax.

In some embodiments, it may be advantageous that the ester wax comprises an ester of a fatty alcohol and/or an ester of a polyol. In some embodiments, the fatty alcohol is as defined above or, alternatively, a C₈-C₄₀-hydrocarbyl alcohol. In some embodiments, the polyol is a C₂-C₈ polyol, in particular of glycol or glycerol. In both cases, the carboxy-group may be formed by a fatty acid. A fatty acid is this sense may in particular be a monovalent linear carboxylic acid having between about 8 and about 24 carbon atoms.

Non-limiting examples of ester waxes include:
i) mono- and di-C₈-C₄₀-hydrocarbyl esters of glycol or glycerol such as glyceryl stearate, glyceryl distearate, glycol stearate and glycol distearate. Such ester waxes are available from SABO S.p.A, Italy.
ii) C₂₀-C₄₀ alkyl (hydroxystearyloxy)stearates. Such waxes are sold, for example, under the trade names “Kester Wax K 82 P” “Hydroxypolyester K 82 P”, “Kester Wax K 80 P”, or “Kester Wax K82H” by the company Koster Keunen BV, Netherlands.
iii) esters of ethylene glycol and montanic acid (octacosanoic acid). Such waxes are sold, for example, under the trade name “Licowax KPS” by the company Clariant AG, Switzerland.
iv) bis(1,1,1-trimethylolpropane) tetrastearate. Such a wax is sold under the name Hest 2T-4S® by the company Heterene Chemical Co, Inc., USA.
v) waxes obtained by hydrogenation of castor oil esterified with cetyl alcohol. Such waxes are sold under the trade names Phytowax ricin 16L64® and 22L73® by the company Sophim SA, France.
vi) beeswax which is a mixture of ester waxes comprising as its main constituents palmitate, palmitoleate, and oleate esters of long-chain (30-32 carbons) aliphatic alcohols, with the ratio of triacontanyl palmitate CH₃(CH₂)₂₉O—CO—(CH₂)₁₄CH₃ to cerotic acid CH₃(CH₂)₂₄COOH, the two principal constituents, being about 6:1.

In some embodiments the solid shaving aid composition may comprise between about 40 and about 85 wt.-%, more specifically between about 55 and about 80 wt.-%, and in particular between about 60 and about 75 wt.-%, of the wax, relative to the total weight of the solid shaving aid composition.

In some embodiments, the wax may be a mixture of waxes.

In some embodiments, the wax comprises a mixture of at least one hydroxylated hydrocarbon-based wax and at least one ester wax.

In some embodiments, the wax comprises a mixture of at least one hydroxylated hydrocarbon-based wax and at least one ester wax, and the relative amount (by weight) of the hydroxylated hydrocarbon-based wax to the amount (by weight) of the ester wax is between about 1:1 to about 20:1, more specifically between about 2:1 to about 15:1, and in particular between about 3:1 to about 10:1.

In some embodiments, the solid shaving aid composition comprises between about 35 and about 80 wt.-%, more specifically between about 37 wt.-% and about 70 wt.-%, and in particular between about 40 wt.-% and about 65 wt.-%, of an hydroxylated hydrocarbon-based wax, and between about 5 and about 25 wt.-%, more specifically between about 6 wt.-% and about 20 wt.-%, and in particular between about 8 wt.-% and about 15 wt.-%, of an ester wax, both relative to the total weight of the solid shaving aid composition. The aforementioned hydroxylated hydrocarbon-based wax and ester wax may independently from each other be selected from the aforementioned disclosure of hydroxylated hydrocarbon-based waxes and ester waxes.

In other embodiments, the wax comprises a polar wax. For the purposes of the present disclosure, the term “polar wax” denotes a wax comprising at least one polar group. Polar groups are well known to those skilled in the art: they may i.a. be selected from hydroxyl, ester, carboxylic acid or amide groups. Such polar waxes are to be differentiated from apolar waxes which are hydrocarbon-based and are substantially free or free of the aforementioned polar groups.

Examples of polar waxes include the aforementioned linear or branched, saturated or unsaturated, monovalent or multivalent hydrocarbyl alcohol having between about 8 and about 32 carbon atoms and the aforementioned ester waxes.

In some embodiments, the wax comprises an apolar wax. Examples of such apolar waxes include polyethylene waxes, paraffin waxes, microcrystalline waxes, ozokerine and Fisher-Tropsch waxes.

As stated above, the solid shaving aid composition comprises at least about 8 wt.-% of a cationic surfactant, relative to the total weight of the solid shaving aid composition.

In some embodiments the cationic surfactant may comprise a cationic surfactant having a positively charged nitrogen-based group, in particular a quaternary ammonium group.

In some embodiments the cationic surfactant may be a saturated or unsaturated hydrocarbon-based compound comprising a quaternary ammonium group and having between about 10 and about 40 carbon atoms, specifically between about 12 and about 36 carbon atoms, and in particular between about 14 about 32 carbon atoms.

In some embodiments the cationic surfactant may comprise a quaternary ammonium group and a saturated or unsaturated alkyl group having between about 8 and about 32 carbon atoms, specifically between about 10 and about 28 carbon atoms, and in particular between about 12 about 26 carbon atoms.

In some embodiments, the aforementioned cationic surfactant may comprise a number of heteroatoms, for instance 1, 2, 3 or 4 oxygen atoms to accommodate ether, keto and ester groups in the molecule.

In some embodiments the cationic surfactant may comprise a quaternary ammonium salt of general formula (I),

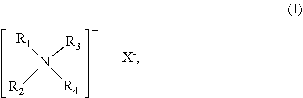

wherein:
X⁻ is an anion selected from halides, such as chloride, bromide and iodide; (C₁-C₆)alkyl sulfates, such as methyl sulfate; phosphates; alkyl, aryl, and alkylaryl sulfonates such as mesylate; and anions derived from carboxylic acids, such as acetate and lactate; or anions of inner salts (e.g. carboxylates);

R₁ and R₂ are each independently from each other selected from optionally substituted linear or branched aliphatic residues comprising between 1 to about 4 carbon atoms and optionally substituted (hetero)aromatic residues such as (hetero)aryl comprising between about 5 to about 12 carbon atoms and alkyl(hetero)aryl residues comprising from about 6 to about 12 carbon atoms; and
R₃ is selected from optionally substituted linear or branched saturated or unsaturated alkyl residues comprising between about 8 and about 32 carbon atoms, specifically between about 10 and about 28 carbon atoms, and in particular between about 12 about 26 carbon atoms; and
R₄ is either defined as R₁ and R₂ or defined as R₃;
or wherein:
X⁻ is an anion selected from halides, such as chloride, bromide and iodide; (C₁-C₆)alkyl sulfates, such as methyl sulfate; phosphates; alkyl, aryl, and alkylaryl sulfonates such as mesylate; and anions derived from carboxylic acids, such as acetate and lactate;
R₁ and R₂ form an optionally substituted saturated, unsaturated, aromatic or heteroaromatic cyclic residue comprising between about 4 to about 26 carbon atoms, more specifically between about 4 and about 10 carbon atoms, and in particular between about 6 to about 16 carbon atoms; and
R₃ is absent and the bond to either R₁ and R₂ is a double bond, or is defined as R₁ and R₂, or represents an optionally substituted linear or branched alkyl residue comprising between 1 and about 6 carbon atoms, in particular between 1 and about 4 carbon atoms; and
R₄ is selected from optionally substituted linear or branched saturated or unsaturated alkyl residues comprising between about 8 and about 32 carbon atoms, specifically between about 10 and about 28 carbon atoms, and in particular between about 12 and about 26 carbon atoms.

Again, in some embodiments, the aforementioned cationic surfactant may comprise a number of heteroatoms, for instance 1, 2, 3 or 4 oxygen atoms to accommodate ether, keto and ester groups in the molecule.

Specific embodiments falling within the formula (I) include lautrimonium salts, myrtrimonium salts, cetrimonium salts, steartrimonium salts, behentrimonium salts, octacosatrimonium salts. Further embodiments, this time including a number of heteroatoms, include palmitoyl carnitine, lauroyl ethyltrimonium salts, myristol ethyltrimonium salts, palmitoyl ethyltrimonium salts, stearoyl ethyltrimonium salts, and cocoylcholine salts.

In some embodiments the cationic surfactant may comprise an imidazolinium salt of general formula (II),

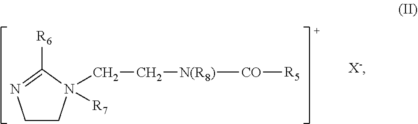

wherein
X⁻ is an anion selected from halides, such as chloride, bromide and iodide; (C₁-C₆)alkyl sulfates, such as methyl sulfate; phosphates; alkyl, aryl, and alkylaryl sulfonates such as mesylate; and anions derived from carboxylic acids, such as acetate and lactate;
R₅ is selected from alkyl and alkenyl residues comprising from about 8 to about 30 carbon atoms, in particular from fatty acid derivatives of tallow;
R₆ is selected from a hydrogen atom, C₁-C₄ alkyl residues, and alkyl and alkenyl residues comprising from about 8 to about 30 carbon atoms;
R₇ is selected from C₁-C₄ alkyl residues;
R₈ is selected from a hydrogen atom and C₁-C₄ alkyl residues.

In some embodiments the cationic surfactant may comprise a diquaternary ammonium salt of general formula (III),

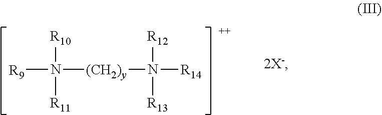

wherein
y is a integer between 1 and about 6, specifically between 1 and about 4, and in particular between about 2 and about 3;
X⁻ is an anion independently selected from halides, such as chloride, bromide and iodide; (C₁-C₆)alkyl sulfates, such as methyl sulfate; phosphates; alkyl, aryl, and alkylaryl sulfonates such as mesylate; and anions derived from carboxylic acids, such as acetate and lactate;
R₉, R₁₀, R₁₁, R₁₂, R₁₃ and R₁₄ are each independently from each other selected from optionally substituted linear or branched aliphatic residues comprising between 1 to about 4 carbon atoms and optionally substituted (hetero)aromatic residues such as (hetero)aryl comprising between about 5 to about 12 carbon atoms and alkyl(hetero)aryl residues comprising from about 6 to about 12 carbon atoms, under the proviso that at least one of R₉, R₁₀, R₁₁, R₁₂, R₁₃ and R₁₄ is selected from optionally substituted linear or branched saturated or unsaturated alkyl residues comprising between about 8 and about 32 carbon atoms, specifically between about 10 and about 28 carbon atoms, and in particular between about 12 about 26 carbon atoms;
or wherein
y is a integer between 1 and about 6, specifically between 1 and about 4, and in particular between about 2 and about 3;
at least two of R₉, R₁₀, R₁₁, R₁₂, R₁₃ and R₁₄ form an optionally substituted saturated, unsaturated, aromatic or heteroaromatic cyclic residue comprising between about 4 to about 24 carbon atoms, more specifically between about 4 and about 10 carbon atoms, and in particular between about 6 to about 16 carbon atoms;
the remainder of R₉, R₁₀, R₁₁, R₁₂, R₁₃ and R₁₄ are each independently from each other selected from optionally substituted linear or branched aliphatic residues comprising between 1 to about 4 carbon atoms and optionally substituted (hetero)aromatic residues such as (hetero)aryl comprising between about 5 to about 12 carbon atoms and alkyl(hetero)aryl residues comprising from about 6 to about 12 carbon atoms, under the proviso that at least one of remainder of R₉, R₁₀, R₁₁, R₁₂, R₁₃ and R₁₄ is selected from optionally substituted linear or branched saturated or unsaturated alkyl residues comprising between about 8 and about 32 carbon atoms, specifically between about 10 and about 28 carbon atoms, and in particular between about 12 about 26 carbon atoms; and
optionally one of the remainder of R₉, R₁₀, R₁₁, R₁₂, R₁₃ and R₁₄ is absent and the bond to one of the at least two of R₉, R₁₀, R₁₁, R₁₂, R₁₃ and R₁₄ forming the optionally substituted saturated, unsaturated, aromatic or heteroaromatic cyclic residue comprising between about 4 to about 26 carbon atoms is a double bond.

Such compounds are sold, for example, under the names Dehyquart® by the company Cognis, Stepanquat® by the company Stepan, Noramium® by the company Ceca, and Rewoquat® WE 18 by the company Evonik.

In some embodiments, the quaternary ammonium salts may be chosen from cetyltrimethylammonium chloride and palmitamidopropyltrimethylammonium chloride sold under the name Varisoft® PA TC. by the company Evonik.

In some embodiments the cationic or zwitterionic surfactant may comprise a polymeric cationic surfactant having a positively charged nitrogen-based group, in particular a quaternary ammonium group. Examples include Polyquaternium-14, Polyquaternium-28, Polyquaternium-32, Polyquaternium-33, Polyquaternium-35, Polyquaternium-36, Polyquaternium-37, Polyquaternium-45, Polyquaternium-47, Polyquaternium-48, Polyquaternium-53, Polyquaternium-63, Polyquaternium-73, Polyquaternium-91, acrylamide/ethyltrimonium chloride acrylate/ethalkonium chloride acrylate copolymers, acrylamidopropyl trimonium chloride/acrylamide copolymers, and acrylamidopropyl-trimonium chloride/acrylates copolymers.

In some embodiments, it may be particularly advantageous that the cationic surfactant comprises behentrimonium methosulfate.

Without wishing to be bound by theory, some of the observed benefits obtained for the solid shaving aid compositions according to the present disclosure may be attributed to the positive charge of the cationic surfactants being able to attach to negative charges of hair or skin proteins. Hereby, cationic surfactants may form a conditioning film on the skin and/or hair stubbles. This conditioning film is believed to provide improved skin hydration, maneuverability and glide. Cationic surfactants may provide such a conditioning film already when present in relatively low concentrations (e.g. as low as 8 wt.-%) compared to other types of surfactants.

Thus, the relative slow release of cationic surfactants from the relatively water-insoluble aforementioned wax-like basis may be seen as one reason why the solid shaving aids of the present disclosure still provide excellent shaving and skin-caring properties despite showing improved durability.

In some embodiments, the solid shaving aid composition may further comprise a zwitterionic surfactant.

In some embodiments the zwitterionic surfactant may be a zwitterionic surfactant having a positively charged nitrogen-based group, in particular a quaternary ammonium group, and a negatively charged group, in particular a sulfonate or carboxylate group.

In some embodiments, the amphoteric surfactants can be selected from aliphatic secondary and tertiary amine derivatives wherein the aliphatic radical is selected from linear and branched chains comprising from about 8 to about 22 carbon atoms and comprising at least one water-soluble anionic group, for example, at least one water-soluble anionic group selected from carboxylate, sulfonate, sulfate, phosphate and phosphonate groups.

In some embodiments zwitterionic surfactant may comprise a betaine and/or a sulfobetaine. In some embodiments the zwitterionic surfactant may comprise a betaine selected from (C₈-C₂₀)alkylbetaines, sulfobetaines, (C₈-C₂₀)alkylamido(C₁-C₆)alkylbetaines; and (C₈-C₁₀)alkylamido(C₁-C₆)alkylsulfobetaines which may optionally be oxyethylenated.

In some embodiments the cationic or zwitterionic surfactant may be pH-insensitive. The pH-insensitivity may refer to the surfactant not changing its ionic charge of its cationic functional groups when the pH is changed within usual physiological ranges (about pH 3.5 to about pH=9). For instance, quaternary ammonium-based surfactants may be pH-insensitive whereas ammonium-based tensides are pH-sensitive since the protonated amino group will change its degree of protonation in the pH-range of about 3.5 to about 9.

In some embodiments the cationic or zwitterionic surfactant may be present in amount of about 8 to about 30 wt.-%, specifically in amounts of about 9 to about at least about 25 wt.-%, and in particular in amounts of about 10 to about 20 wt.-%, relative to the total weight of the solid shaving composition.

In some embodiments the solid shaving aid composition may comprise a mixture of cationic and/or zwitterionic surfactants. However, it has further been found that a cationic surfactant is particularly advantageous. It has been found that cationic surfactants may outperform zwitterionic surfactants during the shaving action, in particular by providing improved glide. Without wishing to be bound by theory, it is believed that this may be due to improved binding of the cationic surfactants to the user's skin. More specifically, cationic surfactants are known to bind to human skin and hair which are typically negatively charged. During the shaving action, the soluted cationic surfactant may bind to the negatively charged skin of the user, as well as the hair. The hydrophobic tail of the cationic surfactant may prevent/reduce the wetting of the skin and hair by water. It should be noted that water itself is a poor lubricant and reducing the wetting of the skin and hair may be considered as providing improved glide. The cationic surfactant may provide this effect more efficiently compared to a zwitterionic surfactant. Without wishing to be bound by theory, it is believed that the cationic surfactant adheres stronger to the user's skin and/or hair compared to zwitterionic surfactants. Cationic surfactants, once attached to the user's skin and/or hair, may be harder to remove by water, compared to zwitterionic surfactants attached to the user's skin and/or hair, which may improve both the repelling of water, as well as the provision of improved glide by the hydrophobic tails. Consequently, in some embodiments, the solid shaving aid composition comprises a cationic surfactant but less than about 3 wt.-%, more specifically less than about 2 wt.-%, and in particular less than about 1 wt.-% of a zwitterionic surfactant. In some embodiments, the weight ratio of cationic surfactant to the zwitterionic surfactant is at least about 3:1, more specifically at least about 4:1 and in particular at least about 5:1. In some embodiments, it may be advantageous that the solid shaving aid composition comprises a cationic surfactant but not a zwitterionic surfactant.

In some embodiments the cationic or zwitterionic surfactant may have a solubility in water of less than about 1000 mg/L, in particular less than about 100 mg/L and in particular less than about 1 mg/L at about 25° C. The solubility may be measured as indicated above with respect to the wax component.

The relative mass ratio of the wax to the cationic or zwitterionic surfactant may be in particular between about 16:1 to about 1:1, more specifically between about 10:1 to about 2:1, and in particular between about 8:1 to about 3:1. Such ratios may help in balancing durability performance and cleansing properties.

In some embodiments, the solid shaving aid composition may comprise, relative to the total weight of the solid shaving aid composition, between about 0.1 wt.-% and about 5 wt.-%, in particular between about 0.5 wt.-% and about 4.5 wt.-%, of a butter or a mixture of two or more butters.

For the purpose of the present disclosure, the term “butter” is intended to mean a lipophilic fatty compound or composition with a reversible solid/liquid change of state, comprising, in its pure form and at a temperature of about 25° C., a liquid and a solid fraction. In other words, the starting melting point of the butter can be less than about 25° C. The liquid fraction of the butter measured at about 25° C. can represent between about 9 wt.-% and about 97 wt.-% of the compound, more specifically about 15 wt.-% and about 85 wt.-%, and in particular between about 40 wt.-% and about 85 wt.-%.

In some embodiments, the butter(s) may have an end melting point of less than about 60° C.

For the purposes of this disclosure, the melting point of a butter may correspond to the temperature of the most endothermic peak observed on thermal analysis (DSC) as described in Standard ISO 1 1357-3:1999. The melting point may e.g. be measured using a differential scanning calorimeter (DSC), for example the calorimeter sold under the name DSC. Q2000 by the company TA Instruments, USA. As regards the measurement of the melting point and the determination of the end melting point, an exemplary sample preparation and measurement protocols may be as follows:

A sample of 5 mg of the butter, preheated to 80° C. and withdrawn with magnetic stirring using a spatula that is also heated, is placed in a hermetic aluminium capsule, or crucible. Two tests are performed to ensure the reproducibility of the results. The measurements are performed on the abovementioned calorimeter. The oven is flushed with nitrogen. Cooling is provided by an RCS 90 heat exchanger. The sample is then subjected to the following protocol: it is first of all placed at a temperature of 20° C., and then subjected to a first temperature rise passing from 20° C. to 80° C., at a heating rate of 5° C./minute, then is cooled from 80° C. to −80° C. at a cooling rate of 5° C./minute and finally subjected to a second temperature rise passing from −80° C. to 80° C. at a heating rate of 5° C./minute. During the second temperature rise, the variation in the difference in power absorbed by the empty crucible and by the crucible containing the sample of butter is measured as a function of the temperature. The melting point of the butter is the value of the temperature corresponding to the top of the peak of the curve representing the variation in the difference in power absorbed as a function of the temperature.

The end melting point corresponds to the temperature at which 95% of the sample has melted.

The liquid fraction by weight of the butter at 25° C. is equal to the ratio of the heat of fusion consumed at 25° C. to the heat of fusion of the butter.

The heat of fusion of the butter is the heat consumed in order to transition from the solid state to the liquid state. The heat of fusion of the butter is equal to the integral of the entire melting curve obtained using the abovementioned calorimeter, with a temperature rise of 5 or 10° C./minute, according to Standard ISO 1 1357-3:1999. The heat of fusion of the butter is the amount of energy required to make the butter change from the solid state to the liquid state. It is expressed in J/g.

The heat of fusion consumed at 25° C. is the amount of energy absorbed by the sample to change from the solid state to the state that it has at 25° C., consisting of a liquid fraction and a solid fraction.

The liquid fraction of the butter measured at 32° C. may represent from 30% to 100% by weight of the butter, more specifically from 50% to 100%, and in particular from 60% to 100% by weight of the compound. When the liquid fraction of the butter measured at 32° C. is equal to 100%, the temperature of the end of the melting range of the butter is less than or equal to 32° C.

The liquid fraction of the butter measured at 32° C. is equal to the ratio of the heat of fusion consumed at 32° C. to the heat of fusion of the butter. The heat of fusion consumed at 32° C. is calculated in the same way as the heat of fusion consumed at 23° C.

In some embodiments, the butter may have a hardness of less than or equal to about 6 MPa. As regards the measurement of the butter hardness, an exemplary sample preparation and measurement protocols may be as follows:

The butter is placed in a mould 75 mm in diameter, which is filled to about 75% of its height. In order to overcome the thermal history and to control the crystallization, the mould is placed in a Votsch VC₀₀₁₈ programmable oven, where it is first of all placed at a temperature of 80° C. for 60 minutes, then cooled from 80° C. to 0° C. at a cooling rate of 5° C./minute, then left at the stabilized temperature of 0° C. for 60 minutes, and then subjected to a temperature rise passing from 0° C. to 20° C., at a heating rate of 5° C./minute, and then left at the stabilized temperature of 20° C. for 180 minutes.

The compressive force measurement is taken using a TA/TX2i texturometer from Swantech. The spindle used is chosen according to the texture:
cylindrical steel spindle 2 mm in diameter for very rigid starting materials;
cylindrical steel spindle 12 mm in diameter for relatively non-rigid starting materials.

The measurement comprises three steps:
a first step after automatic detection of the surface of the sample, where the spindle moves at a measuring speed of 0.1 mm/second, and penetrates into the composition according to the invention disclosure or the butter to a penetration depth of 0.3 mm, and the software notes the maximum force value reached;
a second step, known as relaxation, where the spindle remains in this position for one second and where the force is noted after 1 second of relaxation; and finally
a third step, known as withdrawal, where the spindle returns to its original position at a speed of 1 mm/second, and the withdrawal energy of the probe (negative force) is noted.

The hardness value measured during the first step corresponds to the maximum compressive force measured in newtons divided by the area of the texturometer cylinder expressed in mm² in contact with the butter or the composition according to the disclosure. The hardness value obtained is expressed in megapascals or MPa.

Additionally or alternatively, a butter may be characterized in the solid state (at about 25° C.) by an anisotropic crystal organization, which is visible by X-ray observation. Methods for determining the presence of anisotropic crystal organization within butters are not particularly limited and include XRD and in particular X-ray birefringence imaging (XBI).

In some embodiments, the butter(s) is (are) of plant origin, in particular such as those described in Ullmann's Encyclopedia of Industrial Chemistry (“Fats and Fatty Oils”, A. Thomas, Published Online: 15 Jun. 2000, DOI: 10.1002/14356007.a10_173, point 13.2.2.2. Shea Butter, Borneo Tallow, and Related Fats (Vegetable Butters)) which are incorporated herein by reference.

In some embodiments, the butter(s) comprise butters selected from the following list: Shea butter, Nilotica shea butter (Butyrospermum parkii), galam butter (Butyrospermum parkii), Borneo butter or fat or tengkawang tallow (Shorea stenoptera), shorea butter, illipe butter, madhuca butter or (Bassia) Madhuca longifolia butter, mowrah butter (Madhuca latifolia), katiau butter (Madhuca mottleyana), phulwara butter (M. butyracea), mango butter (Mangifera indica), murumuru butter (Astrocaryum murumuru), kokum butter (Garcinia indica), ucuuba butter (Virola sebifera), tucuma butter, painya (kpangnan) butter (Pentadesma butyracea), coffee butter (Coffea arabica), apricot butter (Prunus armeniaca), macadamia butter (Macadamia ternifolia), grapeseed butter (Vitis vinifera), avocado butter (Persea gratissima), olive butter (Olea europaea), sweet almond butter (Prunus amygdalus dulcis), cocoa butter (Theobroma cacao) and sunflower butter.

In some embodiments, the solid shaving aid composition comprises butter(s) selected from shea butter, cocoa butter and/or mango butter.

In some embodiments, the solid shaving aid composition comprises a mixture of or all of: shea butter, cocoa butter and mango butter.

Butters may help in moisturizing dry and irritated skin. Further, butters may provide skin-soothing and regenerative properties. Furthermore, when in contact with warm skin, butters may (partially) melt and provide a film on the skin, which may provide improved glide and maneuverability to the razor.

In some embodiments the solid shaving aid composition may further comprise a non-ionic surfactant.

In some embodiments, the nonionic surfactants may be chosen from those compounds that are well known per se (see, for example, “Handbook of Surfactants” by M. R. Porter, published by Blackie & Son (Glasgow and London), 1991, pp. 116-178) which are incorporated herein by reference. The nonionic surfactant may in particular be selected from polyethoxylated, polypropoxylated and polyglycerolated fatty acids, alkylphenols, α-diols and alcohols comprising at least one fatty chain comprising, for example, from about 8 to about 18 carbon atoms, it being possible for the number of ethylene oxide or propylene oxide groups to range, for example, from about 2 to about 200 and for the number of glycerol groups to range, for example, from about 2 to about 100.

In some embodiments, the solid shaving aid comprises polyethoxylated, polypropoxylated and polyglycerolated C₈-C₃₀ fatty acid with a polymerization degree of 2 to about 200, more specifically a polyethylene glycolester of C₈-C₃₀ fatty acid, and in particular a polyethylene glycolester of stearic acid with a polymerization degree of 2 to about 200, such as PEG-100 stearate.

The non-ionic surfactants may also be selected from copolymers of ethylene oxide and of propylene oxide, condensates of ethylene oxide and of propylene oxide with C₈-C₃₀-fatty alcohols; polyethoxylated C₈-C₃₀-fatty amines, more specifically, those comprising from about 2 to about 30 mol of ethylene oxide, polyglycerolated C₈-C₃₀-fatty amides comprising on average from about 1 to about 5 glycerol groups, more specifically, from about 1.5 to about 4, glycerol groups; polyethoxylated C₈-C₃₀-fatty amines, more specifically, comprising from 2 to 30 mol of ethylene oxide; oxyethylenated C₈-C₃₀-fatty acid esters of sorbitan comprising from 2 to 30 mol of ethylene oxide; C₈-C₃₀-fatty acid esters of sucrose, C₈-C₃₀-fatty acid esters of polyethylene glycol, C₈-C₃₀-alkylpolyglycosides, and N-C₈-C₃₀-alkylglucamine derivatives.

The non-ionic surfactants may also be selected from alkylpolyglycosides, in particular C₈-C₃₀-alkylpolyglycosides.

In some embodiments the solid shaving aid composition may further comprise a non-ionic surfactant wherein the non-ionic surfactant is not a wax as defined above, in particular not a co-tenside wax.

In some embodiments the solid shaving aid composition may comprise, relative to the total weight of the solid shaving aid composition, less than about 8 wt.-%, more specifically less than about 4 wt.-%, and in particular less than about 2 wt.-%, of the non-ionic surfactant.

Non-ionic surfactants may act as foam enhancers and may reduce skin irritation.

In some embodiments, the solid shaving aid composition may further comprise one or more anionic surfactants. However, in some embodiments, it may be advantageous for compatibility reasons to the cationic surfactants, to keep the amount of such anionic surfactants relatively low. As such, in some embodiments, the solid shaving aid composition may further a total amount of anionic surfactants, relative to the total weight of the solid shaving aid composition, of less than about 10 wt.-%, such as between about 0.1 wt.-% and about 5.0 wt.-%, or between about 0.2 wt.-% and about 2.0 wt.-%.

According to some specific embodiments, the solid shaving aid composition may be substantially free or free of anionic surfactants.

Examples of anionic surfactants include monovalent linear or branched saturated or unsaturated carboxylic acid salts having between about 8 and about 24 carbon atoms, in particular between about 10 and about 20 carbon atoms. Non-limiting examples of such fatty acid salts include salts of cocoate, stearate, palmitate, laurate, myristate, acyl sarcocinate, or mixtures thereof.

In some embodiments, the solid shaving aid composition may comprise, relative to the total weight of the solid shaving aid composition, less than about 10 wt.-%, more specifically less than about 5 wt.-%, and in particular less than about 3 wt.-%, of monovalent linear or branched saturated or unsaturated carboxylic acid salts having between about 8 and about 24 carbon atoms, in particular between about 10 and about 20 carbon atoms.

Further examples of anionic surfactants include sulfur-containing anionic surfactants, in particular anionic surfactants selected from alkyl sulfates, sulfosuccinates, alkyl benzene sulfanate, acyl methyl taurates, isethionates, monoglyceride sulfates and ether sulfanates having between about 8 and about 24 carbon atoms, in particular between about 10 and about 20 carbon atoms. However, in view of their skin irritating potential, the total amount of sulfur-containing anionic surfactants may be selected to be less than about 3.0 wt.-%, such as between about 0.1 wt.-% and about 2.0 wt.-%, or between about 0.2 wt.-% and about 1.5 wt.-%; relative to the total weight of the solid shaving aid composition.

In some embodiments, the solid shaving aid composition may further comprise one or more oils.

Within the present disclosure, the term “oil” refers to C₈-C₅₀-substances that are liquid at room temperature (about 25° C.). These oils may be hydrocarbon-based oils of animal, plant, mineral or synthetic origin, silicone oils and/or fluoro oils, and mixtures thereof. The term “hydrocarbon-based oil” refers to an oil mainly containing carbon atoms and hydrogen atoms (e.g. at least about 70 or about 80 atomic wt.-%).

Specific examples of oils that can be used include:
hydrocarbon-based oils of animal origin, such as perhydrosqualene (squalane);
hydrocarbon-based plant oils, such as liquid triglycerides of fatty acids of 4 to 10 carbon atoms, such as heptanoic or octanoic acid triglyceride; coconut oil, sunflower oil, corn oil, soybean oil, marrow oil, grapeseed oil, sesame oil, hazelnut oil, apricot oil, macadamia oil, arara oil, castor oil, avocado oil, caprylic/capric acid triglycerides, and jojoba oil;
linear or branched hydrocarbons of mineral or synthetic origin, such as liquid paraffin and derivatives thereof, petroleum jelly, polydecenes and hydrogenated polyisobutene, such as parleam;
synthetic esters and ethers, in particular, of fatty acids, such as the oils of formula R₁₅COOR₁₆ in which R₁₅ represents a fatty acid residue containing from about 7 to about 25 carbon atoms and R₁₆ represents a hydrocarbon-based chain containing from about 3 to about 25 carbon atoms, such as, for example, purcellin oil, isononyl isononanoate, isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate, fatty alkyl heptanoates, octanoates or decanoates; polyol esters such as propylene glycol dioctanoate, neopentyl glycol diheptanoate, diethylene glycol diisononanoate; and pentaerythritol esters such as pentaerythrityl tetraisostearate;

In some embodiments, it may be advantageous that the one or more oils comprises a glycerol esterified with three fatty acids. The fatty acids may be as defined above.

In some embodiments, it may be advantageous that the one or more oils comprise one or more oils selected from hydrocarbon-based plant oils, in particular liquid triglycerides of carboxylic acids acids of 4 to 10 carbon atoms; coconut oil, sunflower oil, corn oil, soybean oil, marrow oil, grapeseed oil, sesame oil, hazelnut oil, apricot oil, macadamia oil, arara oil, castor oil, avocado oil, caprylic/capric acid triglycerides, and jojoba oil.

In some embodiments, it may be advantageous that the one or more oils comprises a coconut oil.

In some embodiments, the solid shaving aid composition may comprise, relative to the total weight of the solid shaving aid composition, between about 0.2 wt.-% to about 6 wt.-%, more specifically between about 0.5 wt.-% and about 4 wt.-%, and in particular between about 1.5 wt.-% and about 4 wt.-%, of the one or more oils.

Oils may be added to provide a film on the skin, which may improve the glide and maneuverability of the razor.

In some embodiments the solid shaving aid composition may further comprise a humectant. A humectant may be an agent that retains water in the skin.

In some embodiments, the solid shaving aid composition may comprise, relative to the total weight of the solid shaving aid composition, less than about 4 wt.-%, more specifically less than about 3 wt.-%, and in particular less than about 2 wt.-%, of the humectant.

In some embodiments, the aforementioned one or more oils may comprise one or more humectant oils. The humectant oils may have a molecular weight of greater than about 500 g/mol, more specifically greater than about 700 g/mol, and in particular greater than about 900 g/mol. In particular, the humectant oil may be castor oil derivative, more specifically a castor oil derivative comprising a castor oil esterified with 1, 2 or 3 dicarboxylic acid(s) having between about 3 and about 8 carbon atoms, and in particular castoryl maleate.

In some embodiments, the solid shaving aid composition may comprise, relative to the total weight of the solid shaving aid composition, between about 0.1 wt.-% to about 2 wt.-%, more specifically between about 0.2 wt.-% and about 1 wt.-%, and in particular between about 0.2 wt.-% and about 0.8 wt.-%, of the aforementioned one or more humectant oils having a molecular weight of greater than about 500 g/mol.

In some embodiments, the solid shaving aid composition may comprise, relative to the total weight of the solid shaving aid composition, about 0.1 wt.-% to about 2 wt.-%, more specifically between about 0.2 wt.-% and about 1 wt.-%, and in particular between about 0.2 wt.-% and about 0.8 wt.-%, of the one or more humectant oils having a molecular weight of greater than about 500 g/mol; and, in total, between about 0.2 wt.-% to about 6 wt.-%, more specifically between about 0.5 wt.-% and about 4 wt.-%, and in particular between about 1.5 wt.-% and about 4 wt.-%, of the one or more oils.

The humectant may improve skin hydration. Further, some humectants such as castoryl maleate and its derivatives may contribute to providing a film on the skin which may improve gliding and maneuverability of a razor cartridge.

In some embodiments, the solid shaving aid composition may further comprise one or more monovalent alcohols having a molecular weight of less than about 100 g/mol and/or one or more polyols, in particular polyols having a molecular weight of less than about 250 g/mol. In some instances, such compounds may serve as humectants and/or as processing aids (e.g. as solvent for certain ingredients). In some embodiments, polyols may comprise between about 2 to about 8 hydroxyl groups. In some embodiments, such polyols may comprise from about 2 to about 8 carbon atoms and non-exhaustive examples include glycol, glycerol, pentaerythritol, sorbitol, dipropylene glycol, propylene glycol, butylene glycol, propanediol, ethyl hexanediol, 1,2-hexanediol, and pentylene glycol. In some embodiments, such polyols may comprise from about 12 to about 18 carbon atoms and non-exhaustive examples include disaccharides and trisaccharides such as saccharose, maltose and lactose. Some examples of the aforementioned alcohols includes methanol, ethanol and the propanols.

In some embodiments, in view of keeping the water-solubility of solid shaving aid composition relatively low, it may be advantageous that the solid shaving aid composition comprises, relative to the total weight of the solid shaving aid composition:
a) less than about 10 wt.-%, more specifically less than about 5 wt.-%, and in particular less than about 3 wt.-%, of alcohols and polyols having a molecular weight of less than about 250 g/mol; and/or
b) less than about 10 wt.-%, more specifically less than about 5 wt.-%, and in particular less than about 3 wt.-%, of polyols comprising from about 2 to about 8 carbon atoms; and/or
c) less than about 10 wt.-%, more specifically less than about 5 wt.-%, and in particular less than about 3 wt.-%, of polyols comprising from about 12 to about 18 carbon atoms.

In some embodiments, the total amount of the aforementioned alcohols and polyols may be less than about 10 wt.-%, more specifically less than about 5 wt.-%, and in particular less than about 3 wt.-%, relative to the total weight of the solid shaving aid composition. In some embodiments, the solid shaving aid composition may be substantially free or free of the aforementioned alcohols and/or polyols.

In some embodiments the solid shaving aid composition may further comprise, relative to the total weight of the solid shaving aid composition, less than about 4 wt.-%, more specifically less than about 2 wt.-%, and in particular less than about 0.5 wt.-%, of a silicone compound. However, silicone compounds may accumulate on the skin, which may lead to formation of silicone residues on the user's skin, which may be undesirable. Further, silicone compounds may not be biodegradable. Therefore, it may be preferable to limit the amount of silicone compounds in a solid shaving aid. In some embodiments, the solid shaving aid composition is substantially free or free of silicone compounds.

If silicones are present, it may be advantageous that the solid shaving aid composition comprises, relative to the total weight of the solid shaving aid composition, about 0.1 to about 3 wt.-% of one or more silicone cross-polymers. In some embodiments, the one or more silicone cross-polymers may comprise a first (di)methicone-based polymer chain which is crosslinked to a second (di)methicone-based polymer chain via at least one hydrophilic oligomer/polymer chain. In some embodiments, the first and/or second dimethicone-based polymer chain may be selected from dimethicone and optionally substituted linear or branched saturated or unsaturated C₁-C₂₀ alkyl (di)methicone. In some embodiments, it may be advantageous that the hydrophilic oligomer/polymer chain is a C₁-C₄-alkyl polyether wherein the C₁-C₄-alkyl residues are optionally substituted with hydroxyl, hydroxyl-C₁-C₄-alkyl, C₁-C₄-alkyl ether groups or hydroxyl-C₁-C₄-alkylether groups, in particular polyethylene glycol, polypropylene glycol, polyglycerol, and polysorbate. In some embodiments, the one or more silicone cross-polymer may comprise polyglycerine-modified cross-linked polymers, polyether-modified cross-linked polymers, polyether/alkyl co-modified cross-linked polymers, polyglycerine/alkyl co-modified cross-linked polymers, dimethicone/vinyl dimethicone cross-polymers, dimethicone/phenyl vinyl dimethicone cross-polymers, vinyl dimethicone/lauryl dimethicone cross-polymers, dimethicone crosspolymers, C₃₀-₄₅-alkyl cetearyl dimethicone cross-polymers, cetearyl dimethicone cross-polymers, in particular dimethicone/vinyl dimethicone cross-polymers, vinyl dimethicone/lauryl dimethicone cross-polymers, vinyl dimethicone/lauryl dimethicone cross-polymers, lauryl dimethicone/polyglycerin cross-polymers, dimethicone/polyglycerin cross-polymers, polyethylene glycol/polypropoylene glycol-dimethicone cross-polymer; and mixtures thereof.

In some embodiments the solid shaving aid composition may further comprise an number of generally minor optional ingredients which will be discussed below. It should be understood that the below list is not exhaustive and does not exclude the presence of other ingredients not listed below.

In some embodiments the solid shaving aid composition may comprise, relative to the total weight of the solid shaving aid composition, between about 0.5 wt.-% and about 10.0 wt.-%, in particular between about 1.0 wt.-% and about 8.0 wt.-%, of an opacifier or mixture of opacifiers. In particular, the aforementioned wax glycol distearate may also serve as an opacifier. For the purposes of calculating compositional amounts, glycol distearate will be considered as a wax and not as an opacifier.

In some embodiments the solid shaving aid composition may comprise, relative to the total weight of the solid shaving aid composition, between about 0.1 wt.-% and about 2.5 wt.-%, in particular between about 0.2 wt.-% and about 1.5 wt.-%, of a fragrance or mixture of fragrances.

In some embodiments the solid shaving aid composition may comprise, relative to the total weight of the solid shaving aid composition, between about 0.001 wt.-% and about 2.0 wt.-%, in particular between about 0.005 wt.-% and about 1.0 wt.-%, of an anti-oxidant or mixture of anti-oxidants. Suitable anti-oxidants are not particularly limited and include catechol derivatives such as hydroxymethoxyphenyl decanone.

In some embodiments the solid shaving aid composition may comprise, relative to the total weight of the solid shaving aid composition, between about 0.01 wt.-% and about 2.0 wt.-%, in particular between about 0.05 wt.-% and about 1.0 wt.-%, of a preservative or mixture of preservatives, in particular an antibacterial agent. Examples include benzyl alcohol, phenoxyethanol and dehydroacetic acid.

In some embodiments the solid shaving aid composition may comprise, relative to the total weight of the solid shaving aid composition, less than about 10 wt.-%, more specifically less than about 8 wt.-%, and in particular less than about 6 wt.-%, of water. The water may function as a solvent or processing aid in preparing the solid shaving aid composition.

In some embodiments the solid shaving aid composition may be configured to last for at least about 8 leg shaves or facial shaves, more specifically at least about 9 leg shaves or facial shaves, and in particular at least about 10 leg shaves or facial shaves. The shaves may also be performed on other body areas, such as underarms, bikini etc.

In some embodiments the solid shaving composition may be shaped into the form of a bar, more specifically a bar configured to be placed onto a razor cartridge, and in particular a bar having a length of between about 30 mm to about 80 mm, a width between about 2 mm and about 10 mm, and a depth of about 0.2 mm to about 4 mm.

The solid shaving aid composition having a composition defined as any combination of the above embodiments, may be used in a multiplicity of facial or leg shaving operations, wherein the facial or leg shaving operations is repeated for at least about 8 times, specifically about 9 times, and in particular at least about 10 times.

In some embodiments, the solid shaving aid composition may comprise the following components wherein the components refer to the components as defined above:
a) about 40 to about 85% by weight of one or more waxes;
b) about 8 to about 30% by weight of one or more cationic surfactants,
c) optionally about 0.1 to about 5.0% by weight of one or more butters,
d) optionally about 0.2 to about 6% by weight of one or more oils, further optionally including about 0.1 to about 2.0% by weight, of one or more humectant oils,
e) optionally about 0.5 to about 8% by weight of one or more non-ionic surfactants,
f) optionally about 1.0 to about 10% by weight of water, and
g) optionally up to about 10% by weight, in particular about 0.5 to about 7.5% by weight, of further ingredients, in particular the further ingredients defined above (anionic surfactants, monovalent alcohols, polyols, silicones, opacifiers, fragrances, anti-oxidants, preservatives, etc.) but also other non-mentioned ingredients (e.g. colorants, chelating agents, etc.).

In some embodiments, the solid shaving aid composition may comprise two, three or four of components c) to g). In some embodiments, the solid shaving aid composition may comprise all components c) to g). In some embodiments, the solid shaving aid composition may consist of components a) to g).

In some embodiments, for the purposes of calculating compositional amounts, and in case of an overlap, the compound in question will be allotted according to the category of compound first mentioned in the detailed description. For instance, glycol distearate may be classified as both wax and opacifier but for the purposes of calculating compositional amounts, it will be considered as a wax since the compound category of waxes is mentioned before the opacifiers in the detailed description.

In a second aspect, the present disclosure relates to a solid shaving aid composition. The solid shaving aid composition may have a composition as defined for the first aspect of the present disclosure.

In a third aspect, the present disclosure relates to the use of a solid shaving aid composition having a composition defined above in a multiplicity of facial or leg shaving operations, wherein the facial or leg shaving operations is repeated for at least about 8 times, specifically about 9 times, and in particular at least about 10 times.

Specific embodiments according to the third aspect may use any of the solid shaving aid compositions defined for the first aspect of the present disclosure.

In another related aspect of the present disclosure, there is provided a razor cartridge comprising a housing having a front edge and a rear edge; one or more shaving blades disposed between the front edge and the rear edge; and at least one shaving bar disposed in front of the blade(s) and/or rear of the blade(s) which comprises a solid shaving aid composition; wherein the solid shaving aid composition comprises, relative to the total weight of the solid shaving aid composition: at least about 40 wt.-%, of a wax and at least about 8 wt.-% of a cationic surfactant.

In some embodiments, the solid shaving aid composition may comprise at least about 40 wt.-%, of a wax having a melting point of at least about 40° C. In some embodiments, the solid shaving aid composition may comprise at least about 40 wt.-%, of a wax having a solubility in water at about 25° C. of less than about 1000 mg/L. In some embodiments, the solid shaving aid composition may comprise at least about 40 wt.-%, of a wax having a melting point of at least about 40° C. and a solubility in water at about 25° C. of less than about 1000 mg/L; and at least about 8 wt.-% of a cationic surfactant.

Further embodiments according to this aspect of the disclosure may be as defined for the first aspect of the present disclosure.

### EXAMPLE

A representative composition according to the below table 1 was prepared by hot mixing the components.

TABLE 1
shaving aid compositions

| | Amount |
|---|---|
| Components | (wt.-%) |
| Cationic Surfactant (behentrimonium methosulphate) | 10-20 |
| Hydroxylated Wax (cetearyl alcohol) | 35-65 |
| Ester Waxes (cera alba, glyceryl stearate, glycol | 5-25 |
| distearate) | |
| Non-ionic Surfactant (PEG-100 Stearate) | 0.5-4 |
| Butters (butyrospermum parkii butter, mangifera indica | 0.5-4.5 |
| (mango) seed butter, theobroma cacao (cocoa) seed | |
| butter) | |
| Humectant Oil (castoryl maleate) | 0.2-2 |
| Other Oils (coconut oil, caprylic/capric triglycerides) | 0.1-4 |
| Water | 2.5-6 |
| Other Components (no silicon compound) | 0.01-3 |
| Total | 100 |

The composition was tested by a standard test panel of female users (N>80 and <100). The female panelists were instructed to daily shave their legs and bikini zone with a razor cartridge equipped with a solid shaving aid composition according to above table 1 over a time period of 10 days. The panelists were further asked to evaluate the durability perception as follows: If you had used this razor outside the framework of this study, would you have considered it too worn out to still be used another time? The answers were aggregated to the percentage of panelists on any given day of use willing to use the razor once more.

In a second panel wave, the same panelists were instructed to shave their legs and bikini zone daily with a razor cartridge equipped with a comparative solid shaving aid composition according to WO 2016/162080 A1.

Regarding the durability perception, 63% of the users were willing to use the shaving aid composition again after 10 days (i.e. after 10 uses). In contrast, for the above comparative solid shaving aid composition, less than 30% were willing to use it once more after 10 days. For further comparison, the value of about 63% of users willing to use the razor once more was reached on about day 7 for the comparative solid shaving aid composition.

Although the embodiments of the present disclosure have been disclosed for illustrative purposes, those skilled in the art will appreciate that various modifications and alterations are possible, without departing from the spirit of the present disclosure. It is also to be understood that such modifications and alterations are incorporated in the scope of the present disclosure and the accompanying claims.

While the present disclosure has been primarily directed to various aspects comprising solid shaving aid compositions comprising cationic surfactant, it should be understood that it is in principle possible to replace the cationic surfactant with a zwitterionic surfactant (when the aforementioned compromise on performance is considered to be acceptable). The following list of embodiments do reflect this:
1. A razor cartridge comprising:
a housing having a front edge and a rear edge;
one or more shaving blades disposed between the front edge and the rear edge; and
at least one shaving bar disposed in front of the blade(s) and/or rear of the blade(s) which comprises a solid shaving aid composition;
wherein the solid shaving aid composition comprises, relative to the total weight of the solid shaving aid composition:
at least about 40 wt.-%, of a wax having a melting point of at least about 40° C. and a solubility in water at about 25° C. of less than about 1000 mg/L; and
at least about 8 wt.-% of a cationic or zwitterionic surfactant.
2. The razor cartridge of clause 1, wherein the wax comprises a co-surfactant, in particular a hydroxylated hydrocarbon-based wax.
3. The razor cartridge of clause 1 or clause 2, wherein the wax comprises a linear or branched, saturated or unsaturated, monovalent or multivalent hydrocarbyl alcohol having between about 8 and about 32 carbon atoms, specifically between about 10 and about 28 carbon atoms, and in particular between about 12 about 24 carbon atoms.
4. The razor cartridge of any one of clauses 1 to 3, wherein the wax comprises an optionally linear mono- or divalent alkyl alcohol having between about 8 and about 32 carbon atoms, specifically between about 10 and about 28 carbon atoms, and in particular between about 12 about 24 carbon atoms.
5. The razor cartridge of any one of clauses 1 to 4, wherein the wax comprises a fatty alcohol, in particular cetyl alcohol, stearyl alcohol, behenyl alcohol or mixtures thereof.
6. The razor cartridge of any one of clauses 1 to 5, wherein the wax comprises an ester wax, in particular a hydrocarbon-based ester wax.
7. The razor cartridge of clause 6, wherein the ester wax comprises an ester of a fatty alcohol and/or an ester of a polyol, in particular of glycerol.
8. The razor cartridge of any one of clauses 1 to 7, wherein the solid shaving aid composition comprises between about 40 and about 85 wt.-%, more specifically between about 55 and about 80 wt.-%, and in particular between about 60 and about 75 wt.-%, of the wax, relative to the total weight of the solid shaving aid composition.
9. The razor cartridge of any one of clauses 1 to 8, wherein the cationic or zwitterionic surfactant comprises a cationic surfactant having a positively charged nitrogen-based group, in particular a quaternary ammonium group.
10. The razor cartridge of clause 9, wherein the cationic surfactant is a saturated or unsaturated hydrocarbon-based compound comprising a quaternary ammonium group and having between about 10 and about 40 carbon atoms, specifically between about 12 and about 36 carbon atoms, and in particular between about 14 about 32 carbon atoms.
11. The razor cartridge of clause 9 or clause 10, wherein the cationic surfactant comprises a quaternary ammonium group and a saturated or unsaturated alkyl group having between about 8 and about 32 carbon atoms, specifically between about 10 and about 28 carbon atoms, and in particular between about 12 about 26 carbon atoms.
12. The razor cartridge of any one of clauses 1 to 11, wherein the cationic surfactant comprises a quaternary ammonium salt of general formula (I),

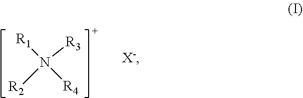

wherein:
X⁻ is an anion selected from halides, such as chloride, bromide and iodide; (C₁-C₆)alkyl sulfates, such as methyl sulfate; phosphates; alkyl, aryl, and alkylaryl sulfonates such as mesylate; and anions derived from carboxylic acids, such as acetate and lactate;
R₁ and R₂ are each independently from each other selected from optionally substituted linear or branched aliphatic residues comprising between 1 to about 4 carbon atoms and optionally substituted (hetero)aromatic residues such as (hetero)aryl comprising between about 5 to about 12 carbon atoms and alkyl(hetero)aryl residues comprising from about 6 to about 12 carbon atoms; and
R₃ is selected from optionally substituted linear or branched saturated or unsaturated alkyl residues comprising between about 8 and about 32 carbon atoms, specifically between about 10 and about 28 carbon atoms, and in particular between about 12 about 24 carbon atoms; and
R₄ is either defined as R₁ and R₂ or defined as R₃;
or wherein:
X⁻ is an anion selected from halides, such as chloride, bromide and iodide; (C₁-C₆)alkyl sulfates, such as methyl sulfate; phosphates; alkyl, aryl, and alkylaryl sulfonates such as mesylate; and anions derived from carboxylic acids, such as acetate and lactate;
R₁ and R₂ form an optionally substituted saturated, unsaturated, aromatic or heteroaromatic cyclic residue comprising between about 4 to about 24 carbon atoms, more specifically between about 4 and about 10 carbon atoms, and in particular between about 6 to about 16 carbon atoms; and
R₃ is absent and the bond to either R₁ and R₂ is a double bond, or is defined as R₁ and R₂, or represents an optionally substituted linear or branched alkyl residue comprising between 1 and about 6 carbon atoms, in particular between 1 and about 4 carbon atoms; and
R₄ is selected from optionally substituted linear or branched saturated or unsaturated alkyl residues comprising between about 8 and about 32 carbon atoms, specifically between about 10 and about 28 carbon atoms, and in particular between about 12 and about 26 carbon atoms.
13. The razor cartridge of any one of clauses 1 to 12, wherein the cationic surfactant comprises an imidazolinium salt of general formula (II),

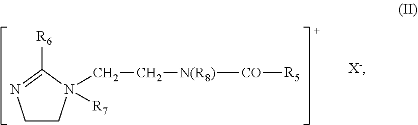

wherein
X⁻ is an anion selected from halides, such as chloride, bromide and iodide; (C₁-C₆)alkyl sulfates, such as methyl sulfate; phosphates; alkyl, aryl, and alkylaryl sulfonates such as mesylate; and anions derived from carboxylic acids, such as acetate and lactate;
R₅ is selected from alkyl and alkenyl residues comprising from about 8 to about 30 carbon atoms, in particular from fatty acid derivatives of tallow;
R₆ is selected from a hydrogen atom, C₁-C₄ alkyl residues, and alkyl and alkenyl residues comprising from about 8 to about 30 carbon atoms;
R₇ is selected from C₁-C₄ alkyl residues;
R₈ is selected from a hydrogen atom and C₁-C₄ alkyl residues.
14. The razor cartridge of any one of clauses 1 to 11, wherein the cationic surfactant comprises a diquaternary ammonium salt of general formula (III),

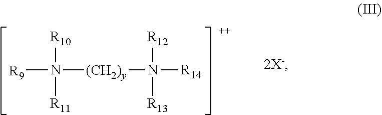

wherein
y is a integer between 1 and about 6, specifically between 1 and about 4, and in particular between about 2 and about 3;
X⁻ is an anion selected from halides, such as chloride, bromide and iodide; (C₁-C₆)alkyl sulfates, such as methyl sulfate; phosphates; alkyl, aryl, and alkylaryl sulfonates such as mesylate; and anions derived from carboxylic acids, such as acetate and lactate;
R₉, R₁₀, R₁₁, R₁₂, R₁₃ and R₁₄ are each independently from each other selected from optionally substituted linear or branched aliphatic residues comprising between 1 to about 4 carbon atoms and optionally substituted (hetero)aromatic residues such as (hetero)aryl comprising between about 5 to about 12 carbon atoms and alkyl(hetero)aryl residues comprising from about 6 to about 12 carbon atoms, under the proviso that at least one of R₉, R₁₀, R₁₁, R₁₂, R₁₃ and R₁₄ is selected from optionally substituted linear or branched saturated or unsaturated alkyl residues comprising between about 8 and about 32 carbon atoms, specifically between about 10 and about 28 carbon atoms, and in particular between about 12 about 26 carbon atoms;
or wherein:
y is a integer between 1 and about 6, specifically between 1 and about 4, and in particular between about 2 and about 3;
X⁻ is an anion selected from halides, such as chloride, bromide and iodide; (C₁-C₆)alkyl sulfates, such as methyl sulfate; phosphates; alkyl, aryl, and alkylaryl sulfonates such as mesylate; and anions derived from carboxylic acids, such as acetate and lactate;
at least two of R₉, R₁₀, R₁₁, R₁₂, R₁₃ and R₁₄ form an optionally substituted saturated, unsaturated, aromatic or heteroaromatic cyclic residue comprising between about 4 to about 24 carbon atoms, more specifically between about 4 and about 10 carbon atoms, and in particular between about 6 to about 16 carbon atoms;
the remainder of R₉, R₁₀, R₁₁, R₁₂, R₁₃ and R₁₄ are each independently from each other selected from optionally substituted linear or branched aliphatic residues comprising between 1 to about 4 carbon atoms and optionally substituted (hetero)aromatic residues such as (hetero)aryl comprising between about 5 to about 12 carbon atoms and alkyl(hetero)aryl residues comprising from about 6 to about 12 carbon atoms, under the proviso that at least one of remainder of R₉, R₁₀, R₁₁, R₁₂, R₁₃ and R₁₄ is selected from optionally substituted linear or branched saturated or unsaturated alkyl residues comprising between about 8 and about 32 carbon atoms, specifically between about 10 and about 28 carbon atoms, and in particular between about 12 about 26 carbon atoms; and
optionally one of the remainder of R₉, R₁₀, R₁₁, R₁₂, R₁₃ and R₁₄ is absent and the bond to one of the at least two of R₉, R₁₀, R₁₁, R₁₂, R₁₃ and R₁₄ forming the optionally substituted saturated, unsaturated, aromatic or heteroaromatic cyclic residue comprising between about 4 to about 24 carbon atoms is a double bond.
15. The razor cartridge of any one of clauses 1 to 12, wherein the cationic surfactant comprises behentrimonium methosulfate.
16. The razor cartridge of any one of clauses 1 to 8, wherein the cationic or zwitterionic surfactant is a zwitterionic surfactant having a positively charged nitrogen-based group, in particular a quaternary ammonium group, and a negatively charged group, in particular a sulfonate or carboxylate group.
17. The razor cartridge of clause 16, wherein zwitterionic surfactant comprises a betaine and/or a sulfobetaine.
18. The razor cartridge of clause 16 or clause 17, wherein the zwitterionic surfactant comprises a betaine selected from (C₈-C₂₀)alkylbetaines, sulfobetaines, (C₈-C₂₀)alkylamido(C₁-C₆)alkylbetaines; and (C₈-C₁₀)alkylamido(C₁-C₆)alkylsulfobetaines which may optionally be oxyethylenated.
19. The razor cartridge of any one of clauses 1 to 18, wherein the cationic or zwitterionic surfactant is pH-insensitive.
20. The razor cartridge of any one of clauses 1 to 19, wherein the cationic or zwitterionic surfactant has a solubility in water of less than about 1000 mg/L, in particular less than about 100 mg/L and in particular less than about 1 mg/L at about 25° C.
21. The razor cartridge of any one of clauses 1 to 20, wherein the cationic or zwitterionic surfactant is present in amount of about 8 to about 30 wt.-%, specifically in amounts of about 9 to about at least about 25 wt.-%, and in particular in amounts of about 10 to about 20 wt.-%, relative to the total weight of the solid shaving composition.
22. The razor cartridge of any one of clauses 1 to 21, wherein the solid shaving aid composition comprises, relative to the total weight of the solid shaving aid composition, less than about 10 wt.-%, more specifically less than about 5 wt.-%, and in particular less than about 3 wt.-%, of monovalent linear or branched saturated or unsaturated carboxylic acid salts having between about 8 and about 24 carbon atoms, in particular between about 10 and about 20 carbon atoms; and/or
less than about 10 wt.-%, more specifically less than about 5 wt.-%, and in particular less than about 3 wt.-%, of monovalent linear or branched saturated or unsaturated carboxylic acids having between about 8 and about 24 carbon atoms, in particular between about 10 and about 20 carbon atoms; and/or
less than about 10 wt.-%, more specifically less than about 5 wt.-%, and in particular less than about 3 wt.-%, of a combination of monovalent linear or branched saturated or unsaturated carboxylic acid salts having between about 8 and about 24 carbon atoms, in particular between about 10 and about 20 carbon atoms, and monovalent linear or branched saturated or unsaturated carboxylic acids having between about 8 and about 24 carbon atoms, in particular between about 10 and about 20 carbon atoms.
23. The razor cartridge of any one of clauses 1 to 22, wherein the solid shaving aid composition comprises, relative to the total weight of the solid shaving aid composition:
a) less than about 10 wt.-%, more specifically less than about 5 wt.-%, and in particular less than about 3 wt.-%, of alcohols and polyols having a molecular weight of less than about 250 g/mol; and/or
b) less than about 10 wt.-%, more specifically less than about 5 wt.-%, and in particular less than about 3 wt.-%, of polyols comprising from about 2 to about 8 carbon atoms; and/or
c) less than about 10 wt.-%, more specifically less than about 5 wt.-%, and in particular less than about 3 wt.-%, of polyols comprising from about 12 to about 18 carbon atoms.
24. The razor cartridge of any one of clauses 1 to 23, wherein the solid shaving aid composition comprises, relative to the total weight of the solid shaving aid composition, less than about 10 wt.-%, more specifically less than about 8 wt.-%, and in particular less than about 5 wt.-%, of water.
25. The razor cartridge of any one of clauses 1 to 24, wherein the solid shaving aid composition comprises, relative to the total weight of the solid shaving aid composition, less than about 4 wt.-%, more specifically less than about 2 wt.-%, and in particular less than about 0.5 wt.-%, of a silicone compound; or wherein the solid shaving aid composition is substantially free or free of silicone compounds.
26. The razor cartridge of any one of clauses 1 to 25, wherein the solid shaving aid composition further comprises a non-ionic surfactant.
27. The razor cartridge of clause 26, wherein the non-ionic surfactant is selected from polyethoxylated, polypropoxylated and polyglycerolated fatty acids; alkylphenols; copolymers of ethylene oxide and of propylene oxide; condensates of ethylene oxide and of propylene oxide with fatty alcohols; polyethoxylated fatty amides; polyglycerolated fatty amides; polyethoxylated fatty amines; oxyethylenated fatty acid esters of sorbitan; fatty acid esters of sucrose; fatty acid esters of polyethylene glycol; and alkylpolyglycosides.
28. The razor cartridge of clause 26 or clause 27, wherein the solid shaving aid composition comprises, relative to the total weight of the solid shaving aid composition, less than about 8 wt.-%, more specifically less than about 8 wt.-%, and in particular less than about 4 wt.-%, of the non-ionic surfactant.
29. The razor cartridge of any one of clauses 1 to 28, wherein the solid shaving aid composition further comprises one or more oils.
30. The razor cartridge of clause 29, wherein the one or more oils comprises a glycerol esterified with three fatty acids, in particular a coconut oil.
31. The razor cartridge of clause 29 or claim 30, wherein the one or more oils comprises one or more humectant oils having a molecular weight of greater than about 500 g/mol, more specifically a castor oil derivative, and in particular castoryl maleate.
32. The razor cartridge of any one of clauses 29 to 31, wherein the solid shaving aid composition comprises, relative to the total weight of the solid shaving aid composition, between about 0.2 wt.-% to about 6 wt.-%, more specifically between about 0.5 wt.-% and about 4 wt.-%, and in particular between about 1.5 wt.-% and about 4 wt.-%, of the one or more oils.
33. The razor cartridge of clauses 31 or 32, wherein the solid shaving aid composition comprises, relative to the total weight of the solid shaving aid composition, between about 0.1 wt.-% to about 2 wt.-%, more specifically between about 0.2 wt.-% and about 1 wt.-%, and in particular between about 0.2 wt.-% and about 0.8 wt.-%, of the one or more humectant oils having a molecular weight of greater than about 500 g/mol.
34. The razor cartridge of any one of clauses 29 to 33, wherein the solid shaving aid composition comprises, relative to the total weight of the solid shaving aid composition, about 0.1 wt.-% to about 2 wt.-%, more specifically between about 0.2 wt.-% and about 1 wt.-%, and in particular between about 0.2 wt.-% and about 0.8 wt.-%, of the one or more humectant oils having a molecular weight of greater than about 500 g/mol; and, in total, between about 0.2 wt.-% to about 6 wt.-%, more specifically between about 0.5 wt.-% and about 4 wt.-%, and in particular between about 1.5 wt.-% and about 4 wt.-%, of the one or more oils.
35. The razor cartridge of any one of clauses 1 to 34, wherein the solid shaving aid composition further comprises, relative to the total weight of the solid shaving aid composition, between about 0.1 wt.-% and about 5 wt.-%, in particular between about 0.5 wt.-% and about 4.5 wt.-%, of a butter or a mixture of butters.
36. The razor cartridge of any one of clauses 1 to 35, wherein the solid shaving aid composition further comprises, relative to the total weight of the solid shaving aid composition, between about 0.1 wt.-% and about 2.5 wt.-%, in particular between about 0.2 wt.-% and about 1.5 wt.-%, of a fragrance or mixture of fragrances.
37. The razor cartridge of any one of clauses 1 to 36, wherein the solid shaving aid composition further comprises, relative to the total weight of the solid shaving aid composition, between about 0.001 wt.-% and about 2.0 wt.-%, in particular between about 0.005 wt.-% and about 1.0 wt.-%, of an anti-oxidant or mixture of anti-oxidants.
38. The razor cartridge of any one of clauses 1 to 37, wherein the solid shaving aid composition is configured to last for at least about 8 leg shaves or facial shaves, more specifically at least about 9 leg shaves or facial shaves, and in particular at least about 10 leg shaves or facial shaves.
39. A solid shaving aid composition having a composition as defined in any one of clauses 1 to 38.
40. The solid shaving composition of clause 39, wherein the solid shaving composition is shaped into the form of a bar, more specifically a bar configured to be placed onto a razor cartridge, and in particular a bar having a length of between about 30 mm to about 80 mm, a width between about 2 mm and about 10 mm, and a depth of about 0.2 mm to about 4 mm.
41. Use of a solid shaving aid composition having a composition as defined in any one of clauses 1 to 38 in a multiplicity of facial or leg shaving operations, wherein the facial or leg shaving operations is repeated for at least about 8 times, specifically about 9 times, and in particular at least about 10 times, in particular without replacing the solid shaving aid composition.

1. A razor cartridge comprising:
a housing having a front edge and a rear edge;
one or more shaving blades disposed between the front edge and the rear edge; and
at least one shaving bar disposed in front of the blade(s) and/or rear of the blade(s) which comprises a solid shaving aid composition;
wherein the solid shaving aid composition comprises, relative to the total weight of the solid shaving aid composition:
at least about 40 wt.-%, of a wax having a melting point of at least about 40° C. and a solubility in water at about 25° C. of less than about 1000 mg/L; and
at least about 8 wt.-% of a cationic surfactant.

2. The razor cartridge of claim 1, wherein the wax comprises a co-surfactant.

3-15. (canceled)

16. The razor cartridge of claim 1, wherein the wax comprises a hydroxylated hydrocarbon-based wax.

17. The razor cartridge of claim 1, wherein the wax comprises an ester wax.

18. The razor cartridge of claim 1, wherein the wax comprises a hydrocarbon-based ester wax.

19. The razor cartridge of claim 1, wherein the cationic surfactant comprises a quaternary ammonium salt of general formula (I), 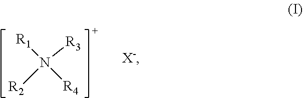
wherein:
X⁻ is an anion selected from halides; (C₁-C₆)alkyl sulfates; phosphates; alkyl, aryl, and alkylaryl sulfonates; and anions derived from carboxylic acids;
R₁ and R₂ are each independently from each other selected from optionally substituted linear or branched aliphatic residues comprising between 1 to about 4 carbon atoms and optionally substituted (hetero)aromatic residues comprising between about 5 to about 12 carbon atoms and alkyl(hetero)aryl residues comprising from about 6 to about 12 carbon atoms; and
R₃ is selected from optionally substituted linear or branched saturated or unsaturated alkyl residues comprising between about 8 and about 32 carbon atoms; and
R₄ is either defined as R₁ and R₂ or defined as R₃;
or wherein:
X⁻ is an anion selected from halides,; (C₁-C₆)alkyl sulfates; phosphates; alkyl, aryl, and alkylaryl sulfonates; and anions derived from carboxylic acids;
R₁ and R₂ form an optionally substituted saturated, unsaturated, aromatic or heteroaromatic cyclic residue comprising between about 4 to about 24 carbon atoms; and
R₃ is absent and the bond to either R₁ and R₂ is a double bond, or is defined as R₁ and R₂, or represents an optionally substituted linear or branched alkyl residue comprising between 1 and about 6 carbon atoms; and
R₄ is selected from optionally substituted linear or branched saturated or unsaturated alkyl residues comprising between about 8 and about 32 carbon atoms.

20. The razor cartridge of claim 1, wherein the solid shaving aid composition comprises behentrimonium methosulfate.

21. The razor cartridge of claim 1, wherein the solid shaving aid composition further comprises a betaine.

22. The razor cartridge of claim 1, wherein the cationic surfactant has a solubility in water of less than about 1000 mg/L.

23. The razor cartridge of claim 1, wherein the solid shaving aid composition comprises, relative to the total weight of the solid shaving aid composition, less than about 10 wt.-%, of monovalent linear or branched saturated or unsaturated carboxylic acid salts having between about 8 and about 24 carbon atoms; and/or
less than about 10 wt.-%, of monovalent linear or branched saturated or unsaturated carboxylic acids having between about 8 and about 24 carbon atoms; and/or
less than about 10 wt.-%, of a combination of monovalent linear or branched saturated or unsaturated carboxylic acid salts having between about 8 and about 24 carbon atoms, and monovalent linear or branched saturated or unsaturated carboxylic acids having between about 8 and about 24 carbon atoms.

24. The razor cartridge of claim 1, wherein the solid shaving aid composition comprises, relative to the total weight of the solid shaving aid composition, less than about 4 wt.-%, of a silicone compound.

25. The razor cartridge of claim 1, wherein the solid shaving aid composition further comprises a non-ionic surfactant.

26. The razor cartridge of claim 1, wherein the solid shaving aid composition further comprises one or more oils.

27. The razor cartridge of claim 1, wherein the solid shaving aid composition further comprises one or more humectant oils having a molecular weight of greater than about 500 g/mol.

28. The razor cartridge of claim 27, wherein the solid shaving aid composition further comprises a castor oil derivative.

29. The razor cartridge of claim 1, wherein the solid shaving aid composition further comprises castoryl maleate.

30. The razor cartridge of claim 1, wherein the solid shaving aid composition further comprises, relative to the total weight of the solid shaving aid composition, between about 0.1 wt.-% and about 5 wt.-% of a butter or a mixture of butters.

31. A solid shaving aid composition comprising, relative to the total weight:
at least about 40 wt.-%, of a wax having a melting point of at least about 40° C. and a solubility in water at about 25° C. of less than about 1000 mg/L; and
at least about 8 wt.-% of a cationic surfactant,
wherein the solid shaving composition is shaped into the form of a bar.

32. A solid shaving aid composition of claim 31 wherein the solid shaving composition is shaped into the form of a bar configured to be placed onto a razor cartridge.

33. A solid shaving aid composition comprising, relative to the total weight:
at least about 40 wt.-%, of a wax having a melting point of at least about 40° C. and a solubility in water at about 25° C. of less than about 1000 mg/L; and
at least about 8 wt.-% of a cationic surfactant,
wherein the solid shaving aid is used in a multiplicity of facial or leg shaving operations, and wherein the facial or leg shaving operations is repeated for at least about 8 times, specifically about 9 times, and in particular at least about 10 times.
